(19) United States
(12) Patent Application Publication
(10) Pub. No.: US 2009/0246870 A1
(43) Pub. Date: Oct. 1, 2009

(54) DE-DIFFERENTIATION OF ASTROCYTES INTO NEURAL STEM CELL USING NANOG

(76) Inventors: Seungkwon You, Gyeonggi-do (KR); Jai Hee Moon, Seoul (KR); Byung Sun Yoon, Seoul (KR); Ki Dong Kim, Gyeonggi-do (KR); Gyuman Park, Gyeonggi-do (KR); Seung Jun Yoo, Gyeonggi-do (KR); Eun Kyung Jun, Gyeonggi-do (KR); Bona Kim, Seoul (KR); Sung Sik Kwak, Incheon (KR); Isaac Maeng, Seoul (KR)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/280,645

(22) PCT Filed: Apr. 12, 2006
(86) PCT No.: PCT/KR2006/001348

§ 371 Date: Oct. 24, 2008

(30) Foreign Application Priority Data
Feb. 27, 2006 (KR) 10-2006-0019012

(51) Int. Cl. C12N 5/02; C12N 5/00

(52) U.S. Cl. 435/377; 435/325

(57) ABSTRACT

Disclosed are a composition and a method for inducing the de-differentiation of astrocytes into neural stem cells using Nanog. The de-differentiated neural stem cells have the ability to differentiate into astrocytes, neurons, or oligodendrocytes.

### TECHNICAL FIELD

Neural stem cells (NSCs) are a subtype of progenitor cells in the nervous system that has the ability to differentiate into astrocytes, oligodendrocytes, and neurons. Originating from the Central Nervous System (CNS) and the Peripheral Nervous System (PNS), neural stem cells form multicellular neurospheres, which differentiate into glial lineage and neural lineage cells under respective sets of conditions (Sally Temple et al. 2001). These neural stem cells are used in the treatment of incurable diseases, and are being studied as a potential method for cell treatment. Extensive research has been conducted on neural stem cells because they are adult stem cells that entail few ethical problems. Active research on de-differentiation, which has been conducted recently, is increasing the importance of adult stem cells. Adult stem cells are easier to obtain than embryonic stem cells, but there are still many difficulties in the practical application thereof. In addition, immune rejection response can be a problem unless using self-originated stem cells. Therefore, the induction of de-differentiation using a patient's own cells could solve current problems. For this purpose, it is necessary to induce de-differentiation of cells that are already differentiated. Currently, extensive research is underway to induce de-differentiation using methods such as cell fusion and nuclear transfer. In another group using different methods, Alexis J. reported that cells isolated from the skin have the characteristics of neural stem cells when cultured under neural stem cell cultivation conditions (Lancet 2004). In addition, Toru K. succeeded in de-differentiating oligodendrocyte precursors into neural stem cells (Genes & Development 2004). This group has been publishing papers on this issue since 2000, and reported in a 2004 paper that gene expression at each stage is relevant to chromatin remodeling and histone modification.

### BACKGROUND ART

In the present invention, a solution to the problems of formerly introduced methods is sought and a novel proper usage method is established. In this invention, one transcription factor which is known to regulate self-renewal, characteristic of embryonic stem cells, is selected and overexpressed to study differentiation into neural stem cells. The selected gene, identified as the “Nanog” gene, is involved in maintaining the plurapotency of embryonic stem cells. As one of the homeodomain protein, a Nanog protein acts as a transcription activator (Kaoru Mitsui, 2003). The Nanog gene is known to be a key factor in regulating the maintenance and differentiation of stem cells. The Nanog gene is expressed abundantly, not only in embryonic stem cells but also in germ cell tumors, however, as mentioned above, it is not expressed in the terminal differentiation stage as the expression amount decreases with the progress of differentiation (Ian Chambers et al., 2003). Since the first disclosure of the Nanog gene in Cell in 2003, a large body of other research followed. Presently, results are being published on the genes regulated by the Nanog gene (Paromita Deb-Rinker et al., 2005, Guangin Pan et al., 2005). In addition, a recent paper published in BBRC indicated that the growth rate of NIH3T3 cells increased with the overexpression of the Nanog gene (Jingyu Ahang et al., 2005).

Leading to the present invention, intensive and thorough research, conducted by the present inventors with this background, resulted in the finding that the overexpression of Nanog therein induces already differentiated astrocytes to de-differentiate into neural stem cell-like cells capable of self-renewal and differentiation into astrocytes, neurons, and oligodendrocytes.

### DISCLOSURE OF THE INVENTION

An object of the invention is to provide a composition for inducing the de-differentiation of astrocytes into neural stem cells, comprising a Nanog protein or a nucleic acid material containing a nucleotide sequence coding for a Nanog protein.

Another object of the invention is to provide a method of inducing the de-differentiation of astrocytes into neural stem cells, comprising the step of treating a Nanog protein or nucleic acid material containing a nucleotide sequence coding for a Nanog protein.

A further object of the invention is to provide neural stem cells which are produced using the method of present invention.

Still a further object of the invention is to provide a method of differentiating the de-differentiated neural stem cells into astrocytes, oligodendrocytes and neurons.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows the overexpression of the Nanog protein, as analyzed through immunocytochemistry;

FIG. 2 shows the induction of de-differentiation, in which sphere formation (A) and direct sphere formation (B) are given (left: phase contrast, right: GFP detection) (Direct spheres are formed when single cells are cultured under conditions for neural stem cells and spheres are formed when single cells are cultured under conditions for neural stem cells only after being attached under conditions for the culture of astrocytes and stabilized for 12 hours);

FIG. 3 shows the expression of neural stem cell markers as analyzed through immunocytochemistry, in which neural stem cells (A to C) and neural stem cell-like cells (Ink4a/Arf^{-/-}astrocyte Nanog #2,#4, p53^{-/-}astrocyte Nanog #4,6) (D to K) are differentiated;

FIG. 4 shows the effect of the Nanog gene on cell growth as measured by growth curve analysis (A) and low density seeding assay (B); and

FIG. 5 shows in vitro differentiation of the neural stem cell-like cells into astrocytes, neurons and oligodendrocytes. (A-C: neural stem cell, D-K: neural stem cell-like cell, D-G: Ink4a/Arf^{-/-}astrocyte Nanog GFP #2,H-J:#4, K:p53^{-/-}astrocyte Nanog GFP#4,L-O:#6)

### BEST MODE FOR CARRYING OUT THE INVENTION

In accordance with an embodiment thereof, the present invention relates to a composition capable of inducing the de-differentiation of astrocytes into neural stem cells, which comprises a Nanog protein or a nucleic acid material containing a nucleotide sequence coding for a Nanog protein.

As used herein, the term “neural stem cell-like cells” is intended to indicate multipotent stem cells, de-differentiated from somatic cells capable of differentiating into neurons, astrocytes and oligodendrocytes. In the present invention, neural stem cell-like cells are also described as neural stem cells.

It is newly disclosed in the present invention that when Nanog is overexpressed, astrocytes, although already differentiated, can de-differentiate into multipotent neural stem cell-like cells.

In the present invention, Nanog is provided in the form of a protein or a nucleic acid that codes for the Nanog protein.

As long as it is originated from mammals such as humans, horses, sheep, pigs, goats, camels, antelopes, and dogs, any Nanog may be used in the composition of the present invention. In addition, the Nanog protein of the present invention, used for de-differentiation into neural cells, may be a wild type pr a variant thereof.

The term “Nanog protein variant” is intended to refer to Nanog proteins, occurring naturally or artificially, which are different in amino acid sequence by one or more amino acids from the wild-type due to the deletion, insertion, non-conservative substitution or conservative substitution of amino acids, or combinations thereof. The variant is a functional equivalent which has the same biological activity as the native protein although its physical and/or chemical properties are modified. Preferably, the variant is increased in structural stability to physical and chemical environments or in physiological activity.

In a preferable embodiment of the present invention, the Nanog is in the form of a nucleic acid material containing a nucleotide sequence coding for the Nanog protein.

The nucleotide sequence coding for the Nanog protein is a nucleotide sequence coding for the wild-type or above mentioned variant type of Nanog proteins, of which one or more bases of the sequence may be varied with the deletion, insertion, non-conservative substitution or conservative substitution of bases, or combinations thereof. This nucleotide sequence can be isolated from the naturally occurring substances or be synthesizes by chemical methods.

The nucleotide sequence that encodes a Nanog protein can be either a single or a double strand consisting of a DNA molecule (genome, cDNA) or an RNA molecule.

In a preferred embodiment of the present invention, a nucleotide sequence coding for a Nanog protein is comprised in a vector that allows a Nanog protein to be expressed.

The term “vector” as used herein, is intended to refer to a DNA construct containing a DNA sequence which is operably linked to a control sequence capable of effecting the expression of the DNA in a suitable host cell.

The term “operably linked” as used herein, is intended to refer to a functional linkage between a nucleic acid sequence regulating gene expression and a second nucleic acid sequence coding for a target protein in such a manner as to enable general functionality. The operable linkage to a recombinant vector may be prepared using a genetic recombinant technique that is well known in the art, and site-specific DNA cleavage and ligation may be carried out using enzymes that are generally known in the art.

A vector suitable for use in the present invention includes a signal sequence or a leader sequence for membrane targeting or secretion as well as expression regulatory elements, such as a promoter, an operator, an initiation codon, a stop codon, a polyadenylation signal and an enhancer, and can be constructed in various forms depending on the purpose thereof. The promoter of the vector may be constitutive or inducible. In addition, expression vectors include a selection marker that allows the selection of host cells containing the vector, and include a replication origin when they are replicable expression vectors. The vector may be self-replicable, or may be integrated into the DNA of a host cell.

The vector useful in the present invention may be a plasmid vector, a cosmid vector, or a viral vector, with preference for a viral vector. Examples of the viral vector includes vectors originated from retroviruses such as HIV (Human Immunodeficiency Virus), MLV (Murine Leukemia Virus), ASLV (Avian Sarcoma/Leukosis Virus), SNV (Spleen Necrosis Virus), RSV (Rous Sarcoma Virus), MMTV (Mouse Mammary Tumor Virus), etc., Adeno-associated viruses, and Herpes Simplex virus, but are not limited thereto.

The nucleic acid material containing the nucleotide sequence coding for a Nanog protein can be introduced into cells in the form of a vector as naked DNA (Wolff et al. Science, 247:1465-8, 1990: Wolff et al. J. Cell Sci. 103:1249-59, 1992), or with the aid of, for example, a liposome or a cationic polymer. For use in gene transfection, a liposome is a phospholipid membrane made of cationic phospholipids such as DOTMA and DOTAP. A cationic liposome, when mixed with a negatively charged nucleotide at a certain ratio, is formed into a nucleic acid-liposome complex.

In another preferred embodiment of the present invention, the nucleic acid material containing a nucleotide sequence that encodes the Nanog protein may be a virus which expresses the Nanog protein therein.

The term “virus”, as used herein, is intended to refer to a Nanog-expressing virus which is prepared by transforming or transfecting a packaging cell with a viral vector carrying a nucleotide sequence coding for the Nanog protein.

Examples of viruses useful in the preparation of the Nanog-expressing viruses according to the present invention include retroviruses, adenoviruses, adeno-associated viruses, and the Herpes Simplex virus, but are not limited thereto. Preferable are retroviruses. In the following examples, a Nanog-expressing virus was prepared by transforming a recombinant pBabe puro vector carrying a nucleotide sequence coding for the Nanog protein (pBabe puro Nanog IRES EGFP) into PT67 packaging cells.

In accordance with another embodiment thereof, the present invention pertains to a method of inducing de-differentiation of astrocytes into neural stem cells, comprising the step of treating a Nanog protein or a nucleic acid material containing a nucleotide sequence for the Nanog protein.

In more detail, the method comprises the steps of (i) culturing astrocytes in a medium; (ii) treating the culture with a Nanog protein or a nucleic acid material containing a nucleotide sequence coding for the Nanog protein; and (iii) inducing the de-differentiation of astrocytes into neural stem cells.

Any conventional culture medium for neural stem cells may be used as a culture medium for astrocytes in step (i). Generally, a culture medium contains a carbon source, a nitrogen source, and trace element ingredients. In addition, the culture medium may include antibiotics, such as penicillin, streptomycin, and gentamicin. Preferred is a culture medium containing bFGF.

The Nanog protein or the nucleic acid material containing the nucleotide sequence coding for the Nanog protein with which the cells are treated in step (ii) is the same as mentioned above.

In accordance with a further embodiment thereof, the present invention pertains to neural stem cells prepared in accordance with the aforementioned method.

It has been confirmed that the neural stem cells, prepared through the de-differentiation according to the present invention, express the neural stem cell-specific markers, Nestin, CD133, and Sox2 at the same level, and have the same ability to differentiate as general neural stem cells. The neural stem cells prepared through de-differentiation according to the present invention feature self-renewal, as well.

In accordance with still a further embodiment thereof, the present invention pertains to a method of differentiating the neural stem cells, de-differentiated according to the aforementioned method, into astrocytes, neurons, and oligodendrocytes.

When the neural stem cells, de-differentiated using the composition and method of the present invention, are placed under respective differentiation conditions which are well-known in the art of field, for astrocyte, neurons, and oligodendrocytes, the differentiation into respective cells can be monitored by detecting the expression of markers specific for respective cells.

A better understanding of the present invention may be obtained through the following examples, which are set forth to illustrate, but are not to be construed as limiting the present invention.

### Example 1

### Experiment Method

1. Cultivation of Ink4a/Arf^{-/-}Astrocytes, p53^{-/-}Astrocytes and Mouse Neural Stem Cells

Ink4a/Arf^{-/-}astrocytes and p53^{-/-}astrocytes were cultured in Dulbecco's modified Eagle's medium (DMEM (high glucose, Hyclone)) supplemented with 10% FBS (HyClone), 1% penicillin/streptomycin, and 1% L-glutamine (Cambrex). Neural stem cells, used as a control, were separated from mice (E13.5) and cultured in Dulbecco's modified Eagle's medium/F12 DMEM/F12, Gibco)(N2), containing insulin (Sigma), apo-transferrin (Sigma), selenium (Sigma), progesterone (Sigma) and penicillin/streptomycin (Cambrex), supplemented with B27 serum-replacement (Gibco), human recombinant basic FGF and human recombinant EGF (R&D).

2. Retroviral-Mediated Infection

The pBabe puro Nanog IRES EGFP (human Nanog (NCBI accession No.: NM_{—}024865) cDNA and IRES EGFP, which is derived from pIRES-EGFP vector were ligated to pBabe puro to construct the pBabe puro Nanog IRES EGFP) or pBabe puro EGFP was transfected into a PT67 packaging cell line (Clontech) using Lipofectamine (Invitrogen) and selected in the presence of puromycin (3 μg/ml) (BD science). After the transformed cell line had grown to 90% or higher confluency, the supernatant was passed through a filter (0.45 μm) (Millipore) to remove cell debris, after which the supernatant was twice allowed to infect the astrocytes, which were separated at intervals of 10 hrs using polybrene (Sigma). Selection was subsequently conducted in the presence of puromycin (0.5 μg/ml).

3. Growth Curve Analysis and Low Density Seeding Assay

12 hours after seeding 1×10⁴ cells per well in 12-well plates, they were stained with 0.01% crystal violet every day for six days. After extraction with acetic acid, density was measured using a spectrophotometer at 600 nm in order to determine the cell growth rate. For the low density seeding assay, 300 cells were seeded per well in 6 well plates and, stained using 0.01% crystal violet to monitor the growth rate after two weeks.

4. Induction of De-Differentiation

De-differentiation was induced under the same culture condition used for neural stem cells. As one method, cell culture was conducted using two different methods. Cells were plated at a density of 1×10⁵ cells/well in 6-well plates and incubated while the medium was replaced with a fresh Dulbecco's modified Eagle's medium/F12 DMEM/F12, Gibco) (N2), supplemented with B27 serum-replacement (Gibco), human recombinant basic FGF and human recombinant EGF (R&D), containing insulin (Sigma), apo-transferrin (Sigma), selenium (Sigma), progesterone (Sigma) and penicillin/streptomycin (Cambrex). Cells were treated with bFGF every day, and the medium was replaced with a fresh medium every other day. Alternatively, cells which had been cultured under proper conditions were trypsinized and then, were seeded at a density of 3×10⁵ cells in a 60-mm bacterial culture plate. Dulbecco's modified Eagle's medium/F12 (DMEM/F12, Gibco), supplemented with B27 serum-replacement (Gibco), human recombinant basic FGF and human recombinant EGF (R&D), containing insulin (Sigma), apo-transferrin (Sigma), selenium (Sigma), progesterone (Sigma) and penicillin/streptomycin (Cambrex), was used as a culture medium.

5. Determination of Overexpression and Respective Markers Through Immunocytochemistry

After fixation with 4% paraformaldehyde (EMS) at 4° C. for one hour, 20% sucrose was added before neurospheres were incubated at 4° C. shaked overnight. Then, the neurospheres was subjected to cryopreservation using an OCT compound in an 8-well chamber slide (Nunc) and was stained after sectioning by 8˜10 μm thick. After blocking with PBS containing 10% normal goat serum (Jackson Immunoresearch)+0.1% BSA (Sigma)+0.3% Triton X-100 (Sigma), the sections were incubated with anti-nestin (Chemicon), anti-CD133 (MACS), anti-Sox2 (Sigma), and anti-Nanog (R&D) at 4° C. overnight and then was subjected to react with anti-mouse-cy3 (Jackson Immunoresearch), anti-rabbit-FITC (Molecular probe), and anti-goat-cy3 (Zymed) at RT, finally followed by nuclear staining with DAPI (Sigma). A Zeiss confocal lens (Carl Zeiss) was used for examination after staining. Differentiated cells were stained in the same manner as described above. In this regard, anti-GFAP (Dako), anti-S100β (Zymed), anti-β-tubulin III (Covance), anti-Map2a (Sigma), anti-TH (Sigma), anti-O4 (R&D), and anti-CNPase (Chemicon) were used as antibodies.

6. In vitro Differentiation

After being coated with PLO (poly-L-ornithine) (Sigma) and laminin or fibronectin (Sigma), cells were cultured under the differentiation conditions given below.

For differentiation into astrocytes, cell culture was conducted for 5˜7 days in DMEM (Hyclone, high glucose) supplemented with 10% FBS (Hyclone) in the presence of human recombinant bFGF and EGF (R&D) or of CNTF (recombinant rat ciliary neurotrophic factor) (Upstate).

Differentiation into neurons was induced by culturing the cells in a N2 medium containing B27(Gibco) serum-replacement, supplemented with human recombinant FGF, for 4 days and then in an FGF-free medium for 8 days. Alternatively, cells were cultured for 7-14 days in the presence of 1˜10 μM of RA (retinoic acid, Sigma). As an additional alternative, cells were cultured for 7˜14 days in the co-presence of 1˜10 Mm of VPA (valproic acid, Sigma) and 1˜10 μM of RA (retinoic acid, Sigma) to induce differentiation into neurons for 20 days.

Differentiation into oligodendrocytes was induced by incubating in an N2 medium supplemented with B27 serum replacement in the presence of PDGF-AA (platelet derived growth factor-AA, R&D), T3 (3,3,5-triiodo-L-thyronine, Sigma), human recombinant basic FGF and EGF (R&D).

While culturing under the aforementioned differentiation conditions, the cells were monitored for morphology, and immunocytochemistry using antibodies specific to respective differentiation markers of cells was conducted to examine resulting differentiation.

### Example 2

### Results

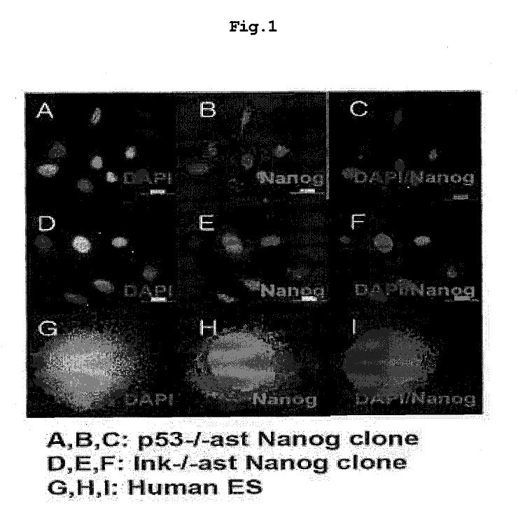

Human Nanog gene was overexpressed using a retroviral transduction system in Ink4a/Arf^{-/-}astrocytes and p53^{-/-}astrocytes, which had been differentiated. The overexpression was verified through immunocytochemistry using a Nanog antibody and reconfirmed by detecting the expression of GFP, positioned just downstream of the gene (FIG. 1).

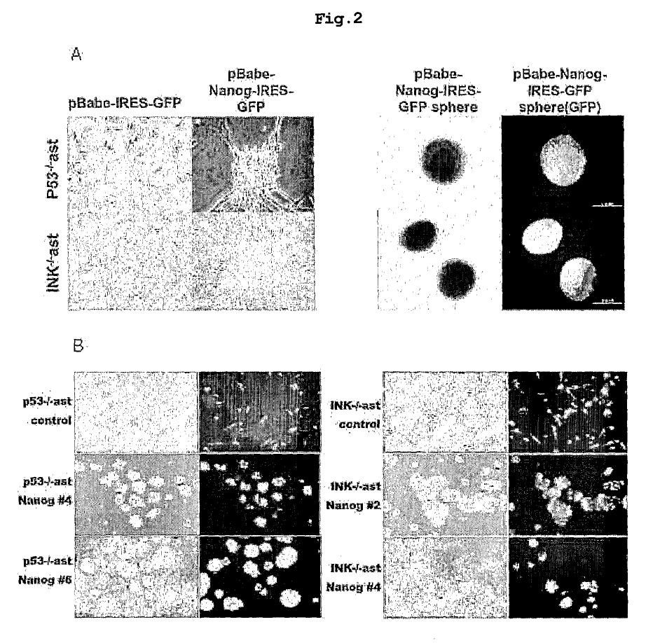

Then, de-differentiation was induced using culture conditions for neural stem cells. The astrocytes were seeded at a density of 1×10⁵ cells per well in 6-well plates, and 12 hrs later, the cells were incubated under conditions suitable for neural stem cells. Within 3-4 days of incubation, the cells were observed to have neural stem cell-like morphology. When the cells were plated at a density of 3×10⁵ cells/plate in 60-mm bacterial plates and cultured under the same conditions as neural stem cells, they were also observed to have the morphology of neural stem cells. On the contrary, when the cells transfected with the control vector pBabe puro EGFP were cultured according to the former method, no neurospheres were formed. In the latter method, neurospheres were observed, but were smaller in size and fewer in number than the cells in which the Nanog gene was expressed (FIG. 2). After one week, the neurospheres were observed to have the same morphology as neural stem cells when they were transferred to and cultured in new plates. In order to examine whether the cells de-differentiated with the Nanog gene ensure self-renewal, a subsphere formation assay was conducted. As a result, spheres were formed in single cells one week later (data not shown).

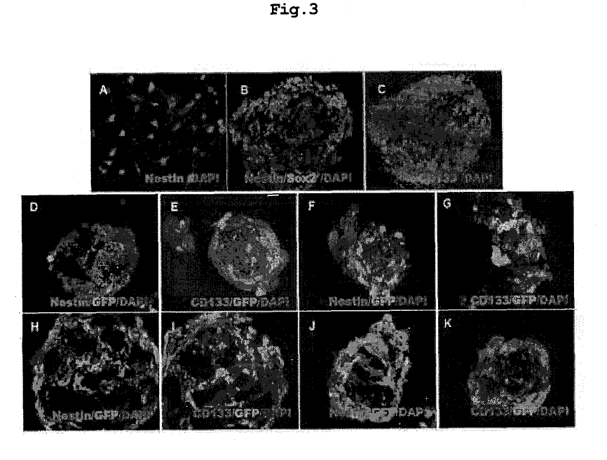

In order to find out whether these cells had the same characteristics as neural stem cells, immunocytochemistry was performed using respective markers. In this regard, after staining, the markers nestin, CD133 and Sox2, which are most abundantly expressed in neural stem cells, were also observed in the same manners as the neural stem cell. Also, the cryo-section of the neurospheres thus formed ensured the expression of nestin, CD133 and Sox2 (FIG. 3).

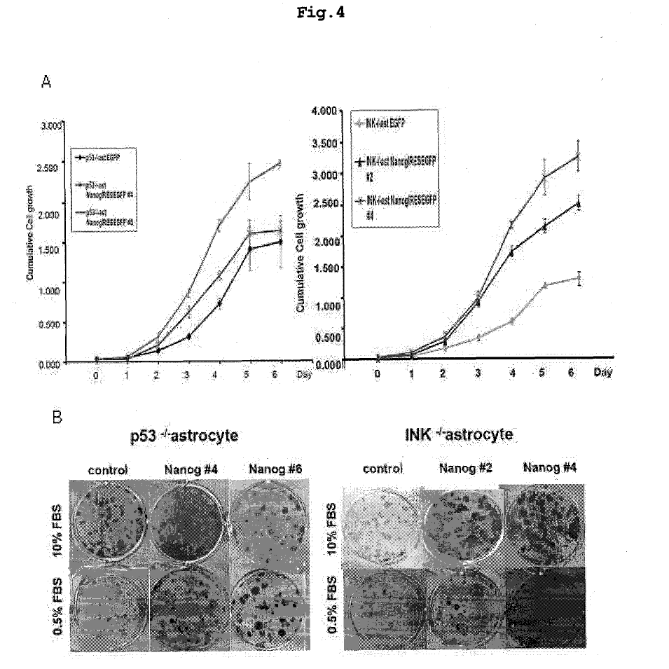

The neurosphere cells thus formed were seeded in 4-well plates coated with PLO and laminin and incubated under cultivation conditions for astrocytes. As a result, six clones were obtained. Two clones showing faster growth rates than the others were selected. The fastest growth rates were observed in #2 and #4 clones when the Nanog gene was overexpressed in Ink4a/Arf^{-/-}astrocytes and in #4 and #6 clones when the Nanog gene was overexpressed in p53^{-/-}astrocytes (FIG. 4).

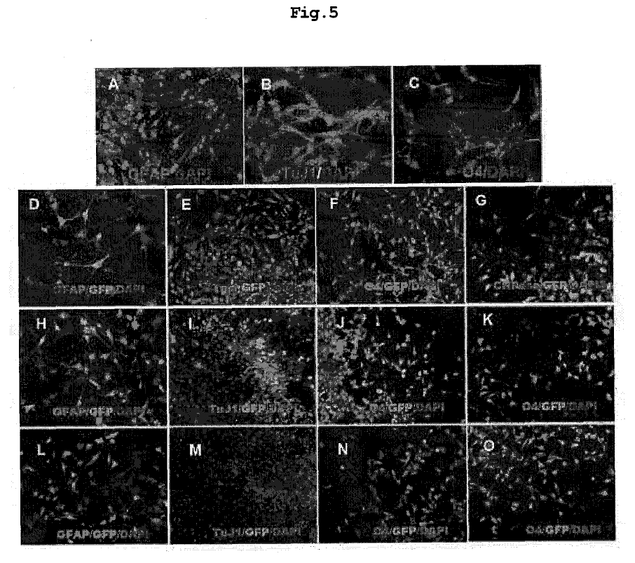

In order to examine whether the neural stem cell-like cells could differentiate like neural stem cells, differentiation into astrocytes, oligodendrocytes and neurons was induced in the same manner as described above. The neural stem cell-like cells were found to differentiate into the three types of cells like neural stem cells as analyzed with antibodies against the respective markers specific therefor. Identification was done with the expression of GFAP and S100 for differentiation into astrocytes, with the expression of β-tubulin III (Tuj1) and Map2a for differentiation into neurons. Also, the expression of O4 and CNPOase for differentiation into oligodendrocytes (FIG. 5) was identified with the differentiation condition thereof.

Taken together, the data obtained through this study demonstrate that the Nanog gene plays a critical role in the de-differentiation of Ink4a/Arf^{-/-}astrocytes and p53^{-/-}astrocytes into neural stem cell-like cells, and that these de-differentiated neural stem cell-like cells can differentiate back into astrocytes, oligodendrocytes, and neurons.

### INDUSTRIAL APPLICABILITY

As described hitherto, Nanog is useful in inducing the de-differentiation of astrocytes into neural stem cells, and the de-differentiated neural stem cells can be used for the treatment of various diseases.

### BIBLIOGRAPHY OF PRIOR ART

1. Toru Kondo, Martin Raff. Chromatin remodeling and histone modification in the conversion of oligodendrocyte precursors to neural stem cells. Genes & Development 2004; 18: 2963-2972
2. Alexis Joannides, Phil Gaughwin, Chistof Shwiening, Henry Majed, Jane Sterling, Alastair Compston, Siddharthan Chandran. Efficient generation of neural precursors from adult human skin: astrocytes promote neurogenesis from skin-derived stem cells. Lancet 2004; 363:172-178
3. Sally Temple. The development of neural stem cells. Nature 2001; 414:112-117
4. Hsieh J, Nakashima K, Kuwabara T, Mejia E, Gage F H. Histone deacetylase inhibitor-mediated neuronal differentiation of multipotent adult neural progenitor cells. PNAS 2004; 101:16659-16664
5. Tarasenko Y I, Yu Y, Jordan P M, Bottenstein J, Wu P. Effect of growth factors on proliferation and phenotypic differentiation of human fetal neural stem cells. Journal of Neuroscience Research 2004; 78:625-636
6. Ping Wu, Yevgeniy Tarasenko, Yanping Gu, Li-Yen M. Huang, Richard E. Coggeshall and Yongjia Yu. Region-specific generation of cholinergic neurons from fetal human neural stem cells grafted in adult rat. Nature neuroscience 2005; 5:1271-1278
7. Hu X, Jin L, Feng L. Erk1/2 but not PI3K pathway is required for neurotrophin 3-induced oligodendrocyte differentiation of post-natal neural stem cells. Journal of Neurochemistry 2004; 90:1339-1347
8. In-Kyung Park, Sean J. Morrison, and Michael F. Clarke. Bmi1, stem cells, and senescence regulation, J. Clin. Invest. 2004; 113:175-179
9. Chambers, D. Colby, M. Robertson, J. Nichols, S. Lee, S. Tweedie and A. Smith, Functional expression cloning of Nanog, a pluripotency sustaining factor in embryonic stem cells. Cell 2003; 113:643-55
10. K. Mitsui, Y. Tokuzawa, H. Itoh, K. Segawa, M. Murakami, K. Takahashi, M. Maruyama, M. Maeda and S. Yamanaka. The homeoprotein Nanog is required for maintenance of pluripotency in mouse epiblast and ES cells. Cell 113 (2003), pp. 631-42
11. T. Kuroda, M. Tada, H. Kubota, H. Kimura, S. Y. Hatano, H. Suemori, N. Nakatsuji and T. Tada. Octamer and Sox elements are required for transcriptional cis regulation of Nanog gene expression. Mol. Cell Biol. 2005; 25: 2475-485

1. A composition for inducing de-differentiation of astrocytes into neural stem cells, wherein astrocytes contain a Nanog protein or the nucleic acid material containing a nucleotide sequence coding for a Nanog protein.

2. The composition as set forth in claim 1, wherein the nucleic acid material containing a nucleotide sequence coding for a Nanog protein is a vector that allows the Nanog protein to be expressed.

3. The composition as set forth in claim 1, wherein nucleic acid material containing a nucleotide sequence coding for a Nanog protein is a virus that expresses the Nanog protein.

4. The composition as set forth in claim 1, wherein the de-differentiated neural stem cells have ability to differentiate into astrocytes, neurons and oligodendrocytes.

5. A method for inducing the de-differentiation of astrocytes into neural stem cells containing the step of treating a Nanog protein or nucleic acid material containing a nucleotide sequence coding for a Nanog protein.

6. The method as set forth in claim 5, wherein the de-differentiated neural stem cells have ability to differentiate into astrocytes, neurons and oligodendrocytes.

7. The method as set forth in claim 5, comprising steps of:
(i) culturing astrocytes in a medium;
(ii) treating a Nanog protein or the nucleic acid material containing nucleotide sequence coding for a Nanog protein; and
(iii) inducing the astrocytes to differentiate into neural stem cells.

8. The method as set forth in claim 7, wherein the medium of step (i) contains bFGF.

9. A neural stem cell, produced using the method of claim 5.

10. A method of differentiating the neural stem cells produced by using the method of claim 5 into astrocytes, neurons, or oligodendrocytes.
